(19) United States
(12) Patent Application Publication
(10) Pub. No.: US 2023/0242428 A1
(43) Pub. Date: Aug. 3, 2023

(54) Remediation of Food Production and Processing Effluents and Waste Products

(71) Applicant: Locus Solutions IPCo, LLC, Solon, OH (US)

(72) Inventors: Sean FARMER, North Miami Beach, FL (US); Ken ALIBEK, Solon, OH (US); Keith HEIDECORN, Solon, OH (US)

(21) Appl. No.: 16/964,980

(22) PCT Filed: Mar. 26, 2020
(86) PCT No.: PCT/US2020/024942

§ 371 Date: Jul. 25, 2020

Related U.S. Application Data
(60) Provisional application No. 62/824,382, filed on Mar. 27, 2019.

(51) Int. Cl. C02F 3/34

(52) U.S. Cl. CPC C02F 3/343; C02F 2101/32

(57) ABSTRACT

The subject invention provides methods for improving the treatment of effluents and waste matter produced during food processing and production. In particular, the subject invention provides methods for remediating fats, oils and greases (FOG), suspended solids, proteins, and other organic matter that are discharged from plants that process, for example, meats, poultry, seafood, dairy and plant-based oils. The methods of the subject invention utilize a customized microbial cocktail comprising facultative anaerobes, in combination with one or more microbial growth by-products, e.g., enzymes and/or biosurfactants, to digest and/or liquefy food processing waste matter.

### CROSS-REFERENCE TO RELATED APPLICATION

This application claims priority to U.S. Provisional Patent Application No. 62/824,382, filed Mar. 27, 2019, which is incorporated herein by reference in its entirety.

### BACKGROUND OF THE INVENTION

The production and processing of food products on an industrial scale results in millions of tons of waste products that, if dispersed untreated into the environment, can be the source of human health hazards, as well as air and water pollution. Fats, oils and greases (FOG), animal carcass trimmings, plant fibers, and other solids or solid-forming substances, are just some of the types of waste products that are left over from the processing of meats, poultry, seafood, dairy, and some plant-based food products. Many of these wastes are stored in lagoons, or large ponds, that emit offensive odors and polluting greenhouse gases. Some food waste products are released into sewers and drains, where they accumulate and cause clogs, as well as into waterways, where they deplete oxygen levels in the water and promote algal blooms.

Attempts have been made to treat food processing wastes using wastewater treatment methods similar to those used in municipal wastewater treatment. In general, “wastewater” is used water from any combination of domestic, municipal, industrial, commercial or agricultural activities, surface runoff, or storm water, as well as any sewer inflow or sewer infiltration. Treatment of wastewater involves multiple processes for removing solid materials, impurities, and contaminants, from the wastewater, including mechanical, chemical and/or biological processes; however, many of these processes are inefficient, making it difficult to keep up with high rates of food waste production.

Meat processing, including, for example, beef, poultry, pork, and other livestock processing, can be a particularly large source of pollution, as well as difficult-to-remediate waste effluents. Prior to processing, live animals are raised in pens or tanks, wherein they release their own metabolic wastes and manure. These wastes, in addition to any oils, hair, feathers and dirt that are washed from the animals during cleaning, end up in wastewater and in ground water as runoff.

Once the animals are slaughtered, they may be de-haired/de-feathered, bled, paunched and washed, further adding these materials to the wastewater stream. Carcasses may be cut, trimmed, and de-boned, with pieces of tissue and bone falling to the floor to be washed away. Further curing and washing of hides adds salts to the wastewater.

Seafood processing can also produce large volumes of polluting effluents, comprising high volumes of FOG, due in part, to the naturally higher fat content of some seafoods. Fish may be washed, sterilized, eviscerated, de-capitated, de-finned, de-boned, de-scaled, skinned, or otherwise processed, leading to solid particles, and fats in the wastewater. Fish, such as tuna, may further be cooked (e.g., via steaming) and canned, leading to oily waste from the steaming condensate, and from the sauces, brines and oils used in the can-filling process.

Along with meat and seafood, dairy processing is a large source of polluting effluents. With dairy products, raw milk in transformed into pasteurized and sour milk, yogurt, hard, soft and cottage cheese, cream and butter products, ice cream, milk powders, lactose, condensed milk, keifers, and dessert products. With the production of cheese and yogurts, for example, whey by-products, including acid whey, can be particularly harmful to aquatic ecosystems. When acid whey, which contains mainly proteins and peptides, enters a stream or other waterway, it depletes the dissolved oxygen levels in the water and its high nutrient content leads to algal blooms. These conditions make it nearly impossible for native fish to survive.

Production and processing of animal-based food products are not the only sources of food waste pollution and wastewater. Production of plant-based oils, for example, palm oil, is a major source of water pollution. Palm oil mill effluent (POME), or the liquid waste that results from the sterilization and clarification process in milling palm oil, contains 90-95% water with the remainder comprising residual oil, soil particles and suspended solids. Due to its high biological oxygen demand (BOD), low pH and colloidal nature, POME can be highly polluting and difficult to remediate.

Furthermore, the post-extraction palm fruits are kept in large lagoons, which bubble and emit a strong odor due to the activity of methanogenic microbes living below the surface. Lagoons are typically utilized as a low-cost method to break down large amounts of organic matter; however, as is the case for palm oil, the organic matter is often converted into greenhouse gases, including carbon dioxide and methane.

Other than lagoons, other forms of food waste treatment utilize anaerobic microbes. For example, after screening out larger solid materials mechanically, anaerobic digesters are often used to digest the remaining solid matter (sludge) and separate out the liquid (water). In an anaerobic digestor, a consortium of microorganisms co-metabolize, or break down, biodegradable material in the absence of oxygen—a process that can take as long as 45 days. As microorganisms utilize the wastewater components to meet their respective nutritional requirements, they produce other chemicals in the process that are beneficial to wastewater treatment and to the metabolic requirements of other beneficial microorganisms. The goal is for these organisms to act collectively to break down the chemical and biological effluents.

The process begins with bacterial hydrolysis of the sludge portion of the wastewater, which often contains insoluble complex organic matter, such as FOG. The insoluble matter is converted into soluble molecules, such as fatty acids, amino acids and sugars. Acidogenic bacteria then convert these compounds into carbon dioxide, hydrogen, ammonia and organic acids, such as acetic acid, butyric acid, propionic acid and ethanol. Methanogens then convert, for example, acetic acid, into methane and carbon dioxide. The remaining indigestible components, such as lignin and non-organic constituents, are then transferred for further treatment or re-used in other ways.

Enzymes and surfactants can also be used for remediating food processing waste. The enzymes primarily serve to attack or degrade organic compounds, while the surfactants act to disperse the degraded particles in the aqueous phase. Some of these compositions, however, have been found to be unstable and yield variable results from one type of waste to another, failing to address the problems presented by waste containing high amounts of various other FOG or fouling substances.

Microbial interactions with wastewater, and with other microorganisms, can be highly effective for treating wastewater. Accordingly, there is a need for a more universal, powerful, and environmentally-friendly microbe-based method for treating food production and processing waste products, such as those discharged from meat, seafood, dairy and plant-oil processing plants.

### BRIEF SUMMARY OF THE INVENTION

In one embodiment, the subject invention provides improved methods for remediating food production and processing effluents and waste products. More specifically, the subject invention provides methods of removing from wastewater, or other bodies of water, an impurity, contaminant or waste matter produced as a result of food processing. The subject invention also provides systems and methods for producing microorganisms and/or their growth by-products, for use in treatment of food processing waste. Advantageously, the methods of the subject invention are environmentally-friendly, operational-friendly and cost effective.

The subject invention provides methods for improving food processing waste treatment, particularly, for bioaugmenting biological wastewater treatment methods.

Treatment, or remediation, of food processing waste products can comprise digesting, purifying, decontaminating, and/or removing waste matter from wastewater. The wastewater can come from, for example, a meat, poultry, or seafood processing plant, a dairy, or facility for milling, handling, extracting and/or refining plant-based oils. The wastewater can contain, for example, organic waste matter such as animal feces, blood, urine and/or stomach contents, carcass remnants, cooking residue, fats, oils and greases (FOG), whey, insoluble polysaccharides and other impurities, such as suspended solids, pathogens, and residue from cleaning of processing plants.

In one embodiment, the methods comprise taking a sample from the wastewater present in an anaerobic digestor, a lagoon, or another body of water into which food processing waste matter has been introduced, wherein the sample comprises food processing waste matter. In some embodiments, the wastewater has been pre-treated to remove large solids, for example, by being passed through a screen, mesh or filter.

The methods can further comprise analyzing the sample to identify the types of waste matter that are present. Based on the types of waste matter that are identified, a customized microbial cocktail is produced, wherein the cocktail comprises a mixture of beneficial microorganisms that are most suitable for the digestion, purification, decontamination and/or removal of the identified waste matter.

In some embodiments, the methods of the subject invention can utilize indigenous microorganisms present in an anaerobic digestor, lagoon or body of water. In some embodiments, the methods can utilize supplemental microorganisms that are not initially present in the digestor, lagoon or body of water.

In certain preferred embodiments, the method utilizes facultative anaerobic bacteria. The microbial cocktail can comprise, for example, different Bacillus spp. microbes, such as, for example, Bacillus spp. bacteria, including, but not limited to, B. subtilis, B. licheniformis, B. firmus, B. laterosporus, B. megaterium, and B. amyloliquefaciens. In some embodiments, the microbes can be Pseudomonas spp. bacteria, such as, for example, P. aeruginosa, P. chlororaphis, P. mallei, P. pseudomallei, P. fluorescens, P. alcaligenes, P. mendocina, and P. stutzeri. Advantageously, in the presence of organic waste matter, these microbes produce enzymes, such as proteases, lipases, reductases and amylases, as well as other growth by-products, which are beneficial to the breakdown of the organic matter.

The microbial cocktail according to the methods of the subject invention can comprise the microorganisms themselves, as well as microbial growth by-products, and any residual growth medium resulting from cultivation of the microbes. The cocktail can further comprise added nutrients for microbial growth.

The microbes can be in the form of vegetative cells, spores, conidia, mycelia and/or a combination thereof. In certain embodiments, the microbes are produced using submerged fermentation, solid-state fermentation (SSF), or combinations and/or modified versions thereof. In preferred embodiments, fermentation is performed using a modified solid state fermentation system. In certain embodiments, the microbial bioaugmentation cocktail is introduced into the wastewater, for example, by pouring the cocktail into the wastewater and mixing it therein. After this point, the microbes in the cocktail grow and/or germinate within the wastewater, producing metabolites to remove impurities, contaminants and/or waste matter therefrom. In some embodiments, germination enhancers can be applied along with the microbial cocktail, particularly if the microbes are applied in spore form. In some embodiments, the process is warmed to increase the rate of removal even further.

In certain embodiments, the wastewater sample further comprises a microbial community. In one embodiment, the sample comprises a representation of the entire microbial community within an anaerobic digestor, lagoon or other body of water into which food processing waste has been introduced.

In some embodiments, the microbial community is analyzed to determine the identity of microbial species present within the microbial community, and to determine the population percentage of each species with respect to the other species of the microbial community. Analysis can comprise standard methods in the art, such as, for example, DNA sequencing, DNA fingerprinting, ELISA, and cell plating.

The species of microbes present in the microbial community can then be categorized as beneficial, commensal or detrimental to the waste treatment process. In some embodiments, the purpose of analyzing the sample is to determine whether the microbial community is in “dysbiosis.” According to the present invention, “dysbiosis” means an overgrowth of commensal and/or detrimental microorganisms, or a microbial community comprising a higher percentage of commensal and/or detrimental microorganisms in relation to the number of beneficial microorganisms.

A wastewater treatment facility that is in dysbiosis is less efficient than one that comprises fewer commensal and/or detrimental microorganisms, meaning the rate of treatment is slower.

A percentage of commensal and/or detrimental microorganisms that is at least 25% of the total population is considered to be dysbiotic. In some embodiments, a dysbiotic microbial community can have more commensal and/or detrimental microorganisms than beneficial microorganisms, or a population percentage greater than 50%.

Upon determining that a microbial community within a sample is dysbiotic, the microbial cocktail can be customized in order to improve the microbial community (i.e., bring the microbial community out of dysbiosis). As a result, the microbial cocktail will bioaugment the speed of the wastewater treatment process (i.e., increase the efficiency of the process using biological means). In certain embodiments, this can also help reduce the amount of nitrous oxide and methane that are produced from wastewater treatment plants by reducing the number of commensal and/or detrimental microbes that produce those compounds.

In one embodiment, the method further comprises introducing a microbial growth by-product that can further enhance the waste treatment process. The growth by-products can include those that are produced by the microbes of the microbial cocktail, or they can be added as a separate component.

In one embodiment, the growth by-products are biosurfactants, enzymes, biopolymers, solvents, acids, proteins, amino acids, or other metabolites that can be useful for remediation of food processing waste matter. In a specific embodiment, the growth by-product is a biosurfactant selected from, for example, low molecular weight glycolipids (e.g., sophorolipids, rhamnolipids, mannosylerythritol lipids and trehalose lipids), lipopeptides (e.g., surfactin, iturin, fengycin, athrofactin and lichenysin), cellobiose lipids, flavolipids, phospholipids (e.g., cardiolipins), and high molecular weight polymers such as lipoproteins, lipopolysaccharide-protein complexes, and polysaccharide-protein-fatty acid complexes.

The one or more biosurfactants can further include any one or a combination of: a modified form, derivative, fraction, isoform, isomer or subtype of a biosurfactant, including forms that are biologically or synthetically modified. In certain embodiments, the one or more biosurfactants are applied in pure form.

Advantageously, the biosurfactants can liquefy certain waste matter, such as solidified FOG, in order to free clogged conduits, as well as increase the flow and drainage of those compounds, and make them more readily accessible for microbial degradation. Additionally, the biosurfactants can work in synergy with enzymes, and/or synergize the different enzymes, that are produced by the microbial cocktail to enhance the treatment of the waste. Furthermore, the biosurfactants are biodegradable.

Advantageously, the methods of the subject invention improve food production and processing waste matter by increasing the proportion of beneficial microorganisms in the treatment environment. Additionally, the microbial population of a particular wastewater treatment system can vary greatly based upon the location of the system and the contents of the waste matter; thus, the methods can accelerate anaerobic processes by utilizing customized groups of organisms that are selectively added to the population to accomplish a narrow range of preferred tasks.

### DETAILED DESCRIPTION

The subject invention provides methods for improving the treatment of effluents and waste matter produced during food processing and production. In particular, the subject invention provides methods for remediating fats, oils and greases (FOG), suspended solids, proteins, and other organic matter that are discharged from plants that process, for example, meats, poultry, seafood, dairy and plant-based oils.

The methods of the subject invention utilize a customized microbial cocktail, in combination with one or more microbial growth by-products, e.g., enzymes and/or biosurfactants, to digest and/or liquefy animal and/or plant-based food processing waste matter. Advantageously, the methods of the subject invention enhance the rate at which digestion, purification, decontamination and/or removal of waste matter occurs in an aerobic digester, lagoon or other body of water into which food processing waste matter has been introduced.

### Selected Definitions

The subject invention utilizes “microbe-based compositions,” meaning compositions that comprise components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites (e.g., biosurfactants), cell membrane components, expressed proteins, and/or other cellular components. The microbes may be intact or lysed. The cells may be totally absent, or present at, for example, a concentration of at least 1×10⁴, 1×10⁵, 1×10⁶, 1×10⁷, 1×10⁸, 1×10⁹, 1×10¹⁰, 1×10¹¹, 1×10¹², or 1×10¹² or more CFU per milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, acids, solvents, alcohols, proteins, carbohydrates, vitamins, minerals, microelements, amino acids, biopolymers, and biosurfactants.

As used herein, the term “plurality” refers to any number or amount greater than one. By “reduces” is meant a negative alteration of at least 1%, 5%, 10%, 25%, 50%, 75%, or 100%.

By “surfactant” is meant a surface active compound that lowers the surface tension (or interfacial tension) between two liquids, between a liquid and a gas, or between a liquid and a solid. Surfactants act as, e.g., detergents, wetting agents, emulsifiers, foaming agents, and dispersants. A “biosurfactant” is a surface-active substance produced by a living cell.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood to be within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Methods of Treating Food Processing Waste

In one embodiment, the subject invention provides improved methods for remediating food production and processing effluents and waste products. More specifically, the subject invention provides methods of removing from wastewater, or other bodies of water, an impurity, contaminant or waste matter produced as a result of food processing. The subject invention also provides systems and methods for producing microorganisms and/or their growth by-products, for use in treatment of food processing waste. Advantageously, the methods of the subject invention are environmentally-friendly, operational-friendly and cost effective.

The subject invention provides methods for improving food processing waste treatment, particularly, for bioaugmenting biological wastewater treatment methods. Preferably, in some embodiments, the methods utilize beneficial microorganisms that produce enzymes and other growth by-products in the presence of organic matter and other waste matter present in wastewater. In certain embodiments, the microbes are facultative anaerobes. In certain embodiments, microbial growth by-products are introduced into the wastewater as a separate and/or combined treatment component with the beneficial microorganisms.

Treatment, or remediation, of food processing waste products can comprise digesting, purifying, decontaminating, and/or removing food processing waste matter present in wastewater. The treatment can be partial and/or it can be complete.

As used herein, “food processing waste products” are waste products that originate from any type of facility used for production, processing, milling, handling, extracting, refining and/or packing of human or animal food commodities. These facilities can include, but are not limited to, slaughterhouses, e.g., for beef, pork, lamb, goat, horse, poultry, and other meat livestock; meat packaging plants; seafood processing plants, e.g., for, farmed or wild-caught fish, shrimp, crawfish, crabs, lobster, scallops, clams, mussels, octopus, squid, and eel; seafood canneries, e.g., for canned tuna or salmon; milking dairies; plants for producing dairy products, e.g., milk, cheese, yogurt, kiefer, and ice cream; cooking oil mills, e.g., for extracting and refining palm oil, olive oil, and other vegetable or fruits oils; and plants where processed foods, such as snacks, novelties, candies, baked goods, and beverages are produced.

Food processing waste products can include, for example, organic waste matter such as animal feces, blood, urine and/or stomach (paunch) contents, carcass remnants (e.g., bones, skin, fur, feathers, fins, nails, teeth, tissue, and organs), cooking residue, fats, oils and greases (FOG), whey, acid-whey, insoluble polysaccharides (e.g., cellulose, lignin) and other impurities, such as suspended solids, deleterious and/or pathogenic microorganisms and residue from cleaning of processing plants. The waste matter can further comprise chemicals and/or condensates from the cleaning, sterilizing, flavoring, dying, or preserving certain processed foods.

In one specific embodiment, the food processing waste products comprise milk whey, which is generated by the processing of dairy products. In one embodiment, the milk whey is acid whey, generated during the production of cottage cheese, cream cheese, as well as Greek and other strained yogurts. Acid whey comprises lactose, water, and a variety of proteins, peptides and lipids.

In one specific embodiment, the food processing waste products comprise palm oil mill effluent (POME), which is generated by palm oil processing mills and comprises suspended components, including oils, oil-bearing cellulosic materials leftover from crushing the palm fruits, and sugars, such as arabinose, xylose, glucose, galactose and mannose.

In one embodiment, the methods comprise taking a sample from the wastewater present in an anaerobic digestor, a lagoon, or another body of water into which food processing waste matter has been introduced (e.g., a pond, stream, lake or river), wherein the sample comprises food processing waste matter. In some embodiments, the wastewater has been pre-treated to remove large solids such as bones and hair, for example, by being passed through a screen, mesh or filter.

The methods can further comprise analyzing the sample to identify the types of waste matter that are present. Based on the types of waste matter that are identified in the sample, a customized microbial cocktail is produced, wherein the cocktail comprises a mixture of beneficial microorganisms that are most suitable for the digestion, purification, decontamination and/or removal of the identified waste matter.

In some embodiments, the methods of the subject invention can utilize indigenous microorganisms present in an anaerobic digestor, lagoon or body of water. In some embodiments, the methods can utilize supplemental microorganisms that are not initially present in the digestor, lagoon or body of water.

The microbial cocktail can comprise, for example, different Bacillus spp. microbes, such as, for example, Bacillus spp. bacteria, including, but not limited to, B. subtilis, B. licheniformis, B. firmus, B. laterosporus, B. megaterium, and B. amyloliquefaciens. In a specific exemplary embodiment, B. amyloliquefaciens NRRL B-67928 is utilized.

In some embodiments, the microbes can be Pseudomonas spp. bacteria, such as, for example, P. aeruginosa, P. chlororaphis, P. mallei, P. pseudomallei, P. fluorescens, P. alcaligenes, P. mendocina, and P. stutzeri.

These microbes can be present in customized ratios. Advantageously, in the presence of organic waste matter, these microbes produce enzymes, such as proteases, lipases, reductases and amylases, as well as other growth by-products, which are beneficial to the breakdown of the organic matter.

Advantageously, in the presence of waste matter, these microbes produce enzymes, such as proteases, lipases, reductases, and amylases, as well as other growth by-products, that are beneficial to the breakdown of food processing waste matter.

In some embodiments, the subject methods can be used for denitrification of wastewater, and/or removing nitrates and/or ammonium from the wastewater and/or activated sludge, wherein the high concentration microbial culture comprises a nitrate-reducing bacteria (NRB), such as, e.g., Thiobacillus denitrificans, Micrococcus spp. (e.g., M denitrificans, M roseus), Serratia spp., Pseudomonas spp., and Achromobacter spp.

The microbial cocktail according to the methods of the subject invention can comprise the microorganisms themselves, as well as microbial growth by-products, and any residual growth medium resulting from cultivation of the microbes. The cocktail can further comprise added nutrients for microbial growth.

Preferably, the microbes of the microbial cocktail are cultivated separately, and the resulting high concentration microbial products are either combined prior to, or at the time of, introducing the cocktail into the wastewater.

The microbes can be in the form of vegetative cells, spores, conidia, mycelia and/or a combination thereof. In certain embodiments, the microbes are produced using submerged fermentation, solid-state fermentation (SSF), or combinations and/or modified versions thereof. In preferred embodiments, fermentation is carried out using a modified solid state fermentation system.

In certain embodiments, the wastewater sample further comprises a microbial community. In one embodiment, the sample comprises a representation of the entire microbial community within an anaerobic digestor, lagoon or other body of water.

The microbial community from the wastewater sample can be analyzed to determine the identity of microbial species present within the microbial community, and to determine the population percentage of each species with respect to the other species of the microbial community. Analysis can comprise standard methods in the art, such as, for example, DNA sequencing, DNA fingerprinting, ELISA, and cell plating.

The species of microbes present in the microbial community can be categorized as beneficial, commensal or detrimental to the water treatment process. In some embodiments, the purpose of analyzing the sample is to determine whether or not the microbial community is in “dysbiosis.” According to the present invention, “dysbiosis” means an overgrowth of commensal and/or detrimental microorganisms, or a microbial community comprising an amount, percentage, or number of commensal and/or detrimental microorganisms greater than the amount, percentage or number of beneficial microorganisms.

As used herein, a “beneficial” microbe is one that confers a benefit to the wastewater treatment process, rather than one that is merely commensal or one that is detrimental. Benefits can include, for example, direct digestion of waste matter in wastewater and/or production of metabolites that help do so.

A “commensal” microorganism is one that exists within the microbial community in a non-beneficial manner, while not necessarily causing any direct harm thereto. Commensal microorganisms can, however, outcompete beneficial microorganisms for space and resources in the wastewater treatment process, thereby causing reduced efficiency. Examples of commensal microorganisms in wastewater treatment can include, for example, Lactobacillus spp., and Bifidus spp.

A “detrimental” microorganism is one that causes direct or indirect harm to the wastewater treatment process, for example, by killing and/or parasitizing beneficial microorganisms or producing harmful growth by-products, including greenhouse gases such as nitrous oxide and methane. Detrimental microorganisms can also include pathogenic organisms, which, if not removed from the wastewater, can cause harm to other living organisms or the environment.

A percentage of commensal and/or detrimental microorganisms that is at least 25%, 30%, 35%, 40%, 45% or higher, of the total population is considered to be dysbiotic. In some embodiments, a dysbiotic microbial community can have more commensal and/or detrimental microorganisms present than beneficial microorganisms, or a population percentage greater than 50%.

A wastewater treatment facility that is in dysbiosis is less efficient than one that comprises fewer commensal and/or detrimental microorganisms. Thus, the subject invention preferably is used to restore a dysbiotic wastewater treatment system to one having a balanced microbial community. A balanced microbial community is one that comprises a variety of microbial species, most of which are beneficial to the wastewater treatment process. For example, in preferred embodiments, at least 50%, 55%. 60%, 65%, 70%, 75% or more, of the microbial community population comprises beneficial microbial species.

Upon determining that a microbial community within a sample is dysbiotic, the method can further comprise producing a customized “microbial cocktail” to add to the microbial community to bring the microbial community out of dysbiosis. As a result, the microbial cocktail will bioaugment the speed of the wastewater treatment process (i.e., increase the efficiency of the process using biological means).

In certain embodiments, this can also help reduce the amount of nitrous oxide and methane (both greenhouse gases) that are produced from anaerobic digesters, lagoons and other water discharge sites, by reducing the number of microbes that produce those compounds. In some embodiments, the methods can be used for reducing the number of deleterious and/or pathogenic microorganisms in wastewater. In some embodiments, the deleterious microorganism is a sulfate-reducing bacteria (SRB) capable of producing harmful hydrogen sulfide gas.

In certain embodiments, the microbial bioaugmentation cocktail is introduced into the wastewater, for example, by pouring the cocktail into the wastewater and mixing it therein. After this point the microbes in the cocktail grow and/or germinate within the wastewater, producing the necessary metabolites to remove impurities, contaminants and/or waste matter therefrom. In some embodiments, germination enhancers can be applied along with the microbial cocktail, particularly if the microbes are applied in spore form. In some embodiments, the process is warmed to increase the efficiency even further.

In one embodiment, the method further comprises introducing a microbial growth by-product that can further enhance the treatment capabilities of the cocktail. The growth by-products can include those that are produced by the microbes of the cocktail, or they can be added as a separate component.

In one embodiment, the growth by-products are biosurfactants, enzymes, biopolymers, solvents, acids, proteins, amino acids, or other metabolites that can be useful for treatment of wastewater. In a specific embodiment, the growth by-product is a biosurfactant.

Biosurfactants are a structurally diverse group of surface-active substances produced by microorganisms. Biosurfactants are biodegradable and can be produced using selected organisms on renewable substrates. Most biosurfactant-producing organisms produce biosurfactants in response to the presence of a hydrocarbon source (e.g., oils, sugar, glycerol, etc.) in the growing media. Other media components such as concentration of iron can also affect biosurfactant production significantly. Microbial biosurfactants are produced by a variety of microorganisms, such as, for example, Pseudomonas spp. (P. aeruginosa, P. putida, P. florescens, P. fragi, P. syringae); Flavobacterium spp.; Bacillus spp. (B. subtilis, B. pumillus, B. licheniformis, B. amyloliquefaciens, B. cereus); Wickerhamomyces spp. (e.g., W anomalus), Candida spp. (e.g., C. albicans, C. rugosa, C. tropicalis, C. lipolytica, C. torulopsis); Rhodococcus spp.; Arthrobacter spp.; Campylobacter spp.; Cornybacterium spp.; Pichia spp. (e.g., P. anomala, P. guilliermondii, P. occidentalis); Starmerella spp. (e.g., S. bombicola); and so on.

All biosurfactants are amphiphiles. They consist of two parts: a polar (hydrophilic) moiety and non-polar (hydrophobic) group. The hydrocarbon chain of a fatty acid acts as the common lipophilic moiety of a biosurfactant molecule, whereas the hydrophilic part is formed by ester or alcohol groups of neutral lipids, by the carboxylate group of fatty acids or amino acids (or peptides), organic acid in the case of flavolipids, or, in the case of glycolipids, by the carbohydrate. Due to their amphiphilic structure, biosurfactants increase the surface area of hydrophobic water-insoluble substances, increase the water bioavailability of such substances, and change the properties of bacterial cell surfaces.

Biosurfactants accumulate at interfaces, thus reducing interfacial tension and leading to the formation of aggregated micellar structures in solution. The ability of biosurfactants to form pores and destabilize biological membranes permits their use as antibacterial, antifungal, and hemolytic agents. Combined with the characteristics of low toxicity and biodegradability, biosurfactants are advantageous for use in a variety of application, including in wastewater treatment.

Biosurfactants according to the subject methods can be selected from, for example, glycolipids (e.g., sophorolipids, rhamnolipids, cellobiose lipids, mannosylerythritol lipids and trehalose lipids), lipopeptides (e.g., surfactin, iturin, fengycin, athrofactin and lichenysin), flavolipids, fatty acid esters, phospholipids (e.g., cardiolipins), and high molecular weight polymers such as lipoproteins, lipopolysaccharide-protein complexes, and polysaccharide-protein-fatty acid complexes.

The one or more biosurfactants can further include any one or a combination of: a modified form, derivative, fraction, isoform, isomer or subtype of a biosurfactant, including forms that are biologically or synthetically modified. In certain embodiments, the one or more biosurfactants are applied in pure form.

Advantageously, the biosurfactants can liquefy certain waste matter, such as solidified FOG, in order to free clogged conduits, as well as increase the flow and drainage of those compounds. Additionally, the biosurfactants can work in synergy with enzymes, and/or synergize the different enzymes, that are produced by the microbial cocktail to enhance the treatment of the waste. Furthermore, the biosurfactants are biodegradable.

Advantageously, the methods of the subject invention increase the efficiency of treating wastewater by increasing the proportion of beneficial microorganisms in the treatment environment. Additionally, the microbial population of a particular wastewater treatment system can vary greatly based upon the location of the system and the contents of the wastewater; thus, the methods can accelerate anaerobic processes by utilizing customized groups of organisms that are selectively added to the population to accomplish a narrow range of preferred tasks. By optimizing the microbial population, a treatment plant can significantly reduce its energy consumption and costs. Furthermore, wastewater treatment does not need to be halted, meaning treatment in accordance with a treatment plant's standard operating procedures can continue uninterrupted during sampling, testing, cultivation, and after introduction of the microbial cocktail according to the subject methods.

### Growth of Microbes According to the Subject Invention

The subject invention provides methods for cultivation of microorganisms and production of microbial metabolites and/or other by-products of microbial growth using a novel form of solid state, or surface, fermentation. Hybrid systems can also be used. As used herein “fermentation” refers to growth of cells under controlled conditions. The growth could be aerobic or anaerobic.

In one embodiment, the subject invention provides materials and methods for the production of biomass (e.g., viable cellular material), extracellular metabolites (e.g., small molecules, polymers and excreted proteins), residual nutrients and/or intracellular components (e.g., enzymes and other proteins).

The microbe growth vessel used according to the subject invention can be any enclosed fermenter or cultivation reactor for industrial use. In one embodiment, the reactor is a proofing oven, such as a standard oven used in commercial baking for, e.g., proofing dough. In one embodiment, the reactor is in the form of a scaled-up enclosure, such as a trailer or a room, that is equipped with the necessary components to provide, for example, tens or hundreds of trays of culture growing on matrix to be incubated at the same time. In one embodiment, the reactor can optionally be equipped with an automated conveyor system or pulley system for continuous production.

In one embodiment, the vessel may optionally have functional controls/sensors or may be connected to functional controls/sensors to measure important factors in the cultivation process, such as pH, oxygen, pressure, temperature, agitator shaft power, humidity, viscosity and/or microbial density and/or metabolite concentration. Preferably, no such controls are necessary, however.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of microbes in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. The oxygenated air may be ambient air supplemented daily through, e.g., air pumps.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors, trace nutrients and/or biostimulants for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate (e.g., ferrous sulfate heptahydrate), iron chloride, manganese sulfate, manganese sulfate monohydrate, manganese chloride, zinc sulfate, lead chloride, copper sulfate, calcium chloride, calcium carbonate, and/or sodium carbonate. These inorganic salts may be used independently or in a combination of two or more.

In some embodiments, when, for example, the microbes used to inoculate the substrate are in spore form (e.g., bacterial endospores), germination enhancers can be added to the substrate. Examples of germination enhancers according to the subject invention include, but are not limited to, L-alanine, manganese, L-valine, and L-asparagine or any other known germination enhancer.

In some embodiments, the method for cultivation may optionally comprise adding additional acids and/or antimicrobials in to the substrate before and/or during the cultivation process. Advantageously, however, the subject method reduces or eliminates the need for protection from contamination during cultivation due in part to the slower rate of microbial growth.

The, pH of the mixture should be suitable for the microorganism of interest, though advantageously, stabilization of pH using buffers or pH regulators is not necessary when using the subject cultivation methods.

The method and equipment for cultivation of microorganisms and production of the microbial by-products can be performed in a batch process or a quasi-continuous process.

In one embodiment, the method for cultivation of microorganisms is carried out at about 15 to 60° C., preferably, 25 to 40° C., and in specific embodiments, 25 to 35° C., or 32 to 37° C. In one embodiment, the cultivation may be carried out continuously at a constant temperature. In another embodiment, the cultivation may be subject to changing temperatures. Temperature can be kept within the preferred range by pumping ambient air into the reactor and circulating it throughout.

In one embodiment, total sterilization of equipment and substrate used in the subject cultivation methods is not necessary. However, the equipment and substrate can optionally be sterilized. The trays can be sterilized before and/or after being spread with nutrient medium, for example, using an autoclave. Additionally, the steam pan lids and pan bands can be sterilized, for example, by autoclaving, prior to inoculation of the solid substrate.

The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control bacterial growth.

In one embodiment, the subject invention further provides methods of producing a microbial metabolite by cultivating a microbe strain under conditions appropriate for growth and metabolite production. Optionally, the method can comprise purifying the metabolite. The subject invention provides methods of producing metabolites such as, e.g., biosurfactants, biopolymers, ethanol, lactic acid, beta-glucan, proteins, peptides, metabolic intermediates, polyunsaturated fatty acid, lipids and enzymes.

The microbial growth by-product produced by microorganisms of interest may be retained in the microorganisms or secreted into the substrate. The metabolite content can be, for example, at least 20%, 30%, 40%, 50%, 60%, 70%, 80%, or 90%.

In another embodiment, the method for producing microbial growth by-product may further comprise steps of concentrating and purifying the microbial growth by-product of interest. In a further embodiment, the substrate may contain compounds that stabilize the activity of microbial growth by-product.

In one embodiment, all of the microbial cultivation composition is removed upon the completion of the cultivation (e.g., upon, for example, achieving a desired spore density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a cell-free substrate or contain cells. In this manner, a quasi-continuous system is created.

### Matrix Fermentation

In preferred embodiments, the subject invention provides methods for cultivating microbe-based products using novel procedures and systems for solid state, or surface, fermentation. Advantageously, the subject invention does not require fermentation systems having sophisticated aeration systems, mixers, or probes for measuring and/or stabilizing DO, pH and other fermentation parameters.

In preferred embodiments, the method of cultivating a microorganism and/or producing a microbial growth by-product comprises: spreading a layer of a solid substrate mixed with water and, optionally, nutrients to enhance microbial growth, onto a tray to form a matrix; applying an inoculant of the desired species onto the surface of the matrix; placing the inoculated tray into a fermentation reactor; passing air through the reactor to stabilize the temperature between 25-40° C.; and allowing the microorganism to propagate throughout the matrix.

In preferred embodiments, the matrix substrate according to the subject methods comprises foodstuffs. The foodstuffs can include, for example, rice, beans or legumes, corn and other grains, pasta, wheat bran, flours or meals (e.g., corn flour, nixtamilized corn flour, partially hydrolyzed corn meal), and/or other similar foodstuffs to provide surface area for the microbial culture to grow and/or feed on.

In one embodiment, wherein the matrix substrate comprises pre-made pasta, the pasta can be made from, for example, corn flour, wheat flour, semolina flour, rice flour, quinoa flour, potato flour, soy flour, chickpea flour and/or combinations thereof. In some embodiments, the pasta is made from an enriched flour.

In some embodiments, the pasta can be in the shape of a long string or ribbon, e.g., spaghetti or fettuccini. In some embodiments, the pasta can be in the shape of, for example, a sheet, a shell, a spiral, a corkscrew, a wheel, a hollow tube, a bow, or any variation thereof. Advantageously, the microbes can grow inside the pasta and/or on outside surfaces of the pasta. This increases the surface area upon which the microorganisms can grow, increases the depth of microbial growth within the substrate, and provides enhanced oxygen penetration within the culture.

In one embodiment, wherein the matrix substrate comprises grains of rice, the matrix substrate can be prepared by mixing rice grains with water and, depending upon which microbe is being cultivated, an added nutrient medium.

In one embodiment, the method of cultivation comprises preparing the trays, which can be, e.g., metal sheet pans or steam pans fitted for a standard proofing oven. In some embodiments, the “trays” can be any vessel or container capable of holding the substrate and culture, such as, for example, a flask, cup, bucket, plate, pan, tank, barrel, dish or column, made of, for example, plastic, metal or glass.

Preparation can comprise covering the inside surfaces of the trays with, for example, foil. Preparation can also comprise sterilizing the trays by, for example, autoclaving them.

Next, a matrix substrate is prepared by mixing a foodstuff item, water, and optionally, additional salts and/or nutrients to support microbial growth. In a specific embodiment, the nutrient medium can comprise, for example, maltose, yeast extract or another source of protein, and sources of minerals, potassium, sodium, phosphorous and/or magnesium.

The mixture is then spread onto the trays and layered to form a matrix with a thickness of approximately 1 to 12 inches, preferably, 1 to 6 inches. The thickness of the matrix can vary depending on the volume of the tray or other container in which is it being prepared.

In preferred embodiments, the matrix substrate provides ample surface area on which microbes can grow, as well as enhanced access to oxygen supply. Thus, the substrate on which the microbes grow and propagate can also serve as the nutrient medium for the microbes.

In some embodiments, grooves, ridges, channels and/or holes can be formed in the matrix to increase the surface area upon which the microorganisms can grow. This also increases the depth of microbial growth within the substrate and provides enhanced oxygen penetration throughout the culture.

To increase microbial motility throughout the substrate, the method can further comprise applying a biostimulant, potato extract and/or banana peel extract to the substrate. This allows for increased speed of distribution of the culture throughout the surfaces of the substrate.

In some embodiments, when, for example, the microbes used to inoculate the substrate are in spore form, germination enhancers can be applied to the substrate. Examples of germination enhancers according to the subject invention include, but are not limited to, L-alanine, manganese, L-valine, and L-asparagine or any other known germination enhancer.

Sterilization of the trays and matrix can then be performed after the matrix has been spread onto the trays. Sterilization can be performed by autoclave or any other means known in the art. In some embodiments, this process will also effectively cook the substrate.

Lids and silicon pan bands can be provided for sealing the trays, if desired. To create a completely sterile system, the lids and pan bands can also be sterilized.

After preparing the matrix substrate in the trays, the trays can be inoculated with a desired microorganism that is optionally pre-mixed with sterile nutrient medium. Optionally, depending upon the microorganism being cultivated and/or the growth by-product being produced, the trays can then be sealed with the lids and pan bands. In one embodiment, for example, when the microorganism is a Bacillus spp. bacteria, the trays are preferably not sealed.

The inoculum can comprise vegetative cells, spores or other forms of the microorganism. In one embodiment, inoculation is performed by applying the inoculum uniformly onto the surface of the substrate layer. The inoculum can be applied via, for example, spraying, sprinkling, pouring, injecting or spreading. In one embodiment, inoculation is carried out using a pipette.

The inoculated trays can then be placed inside a fermentation reactor. In one embodiment, the trays are placed inside a proofing oven. The proofing oven can be, for example, a standard proofing oven used in commercial baking. Optionally, the reactor can be equipped with a conveyer system, wherein the trays move continuously through the reactor using, for example, a conveyer belt or a pulley system.

In one embodiment, a plurality of reactors can be used, for example, a plurality of proofing ovens. In one embodiment, the reactors are distributable and portable. In a further embodiment, wherein a plurality of reactors is used, the plurality of reactors can be assembled onto a single platform for ease of transport.

Fermentation parameters can be adjusted based on the desired product to be produced (e.g., the desired microbial biosurfactant) and the microorganism being cultivated.

The temperature within the reactor depends upon the microorganism being cultivated, although in general, it is kept between about 25-40° C. using ambient air pumped through the reactor. The circulating air can also provide continuous oxygenation to the culture. The air circulation can also help keep the DO at desired levels, for example, about 90% of ambient air.

In one embodiment, it is not necessary to monitor or stabilize the pH of the culture. The trays may be sprayed regularly throughout fermentation (e.g., once a day, once every other day, once per week) with a sterile nutrient medium for achieving maximum microbial concentration.

The culture can be incubated for an amount of time that allows for the microorganism to reach a desired concentration, or to reach from 50-100% sporulation, preferably from 1 day to 14 days, more preferably, from 2 days to 10 days.

In some embodiments, the microorganisms will consume either a portion of, or the entirety of, the matrix substrate throughout fermentation.

Once the culture sporulates, the culture and remaining substrate can be harvested from the trays, then blended together to produce a microbial slurry. The concentration of microbes grown according to this method can reach, for example, 1×10⁶ to 1×10¹³ propagules (or CFU) per gram, preferably 1×10⁸ to 1×10¹³ CFU/g, or at least 5×10⁹ to 5×10¹⁰ CFU/mL when dissolved in water.

In one embodiment, the microbial slurry is milled, micronized and/or dried to produce a dry microbe-based product that contains the microorganism, its growth by-products and matrix substrate. The microbial slurry can be dried using any drying method known in the art. In one embodiment, the dried product has approximately 3% to 6% moisture retention.

In one embodiment, the solution containing the dissolved culture is diluted to a concentration of 1×10⁶ to 1×10⁷ CFU/mL using water to form a liquid microbe-based product, which can be utilized in a wide variety of settings and applications. Optionally, nutrients including, e.g., sources of potassium, phosphorous, magnesium, carbon, proteins, amino acids, and others can be added to the water to enhance microbial growth.

Activation and/or germination of spore-form microbes can be enhanced, either during cultivation or at the time of application of the microbe-based product, by adding L-alanine in low (micromolar) concentrations, manganese or any other known germination enhancer.

In one embodiment, the systems and methods of the subject invention can be used to produce a microbial metabolite, wherein instead of drying the microbial slurry, the microbial slurry is filtered to separate the liquids from the solids. The liquid that is extracted, which comprises the microbial metabolite, can then be purified further, if desired, using, for example, centrifugation, rotary evaporation, microfiltration, ultrafiltration and/or chromatography.

The metabolite and/or growth by-product can be, for example, a biosurfactant, enzyme, biopolymer, acid, solvent, amino acid, nucleic acid, peptide, protein, lipid and/or carbohydrate. Specifically, in one embodiment, the method can be used to produce a biosurfactant.

Advantageously, the method does not require complicated equipment or high energy consumption. The microorganisms of interest can be cultivated at small or large scale on site and utilized, even being still-mixed with their media. Similarly, the microbial metabolites can also be produced at large quantities at the site of need.

Advantageously, the microbe-based products can be produced in remote locations. The microbe growth facilities may operate off the grid by utilizing, for example, solar, wind and/or hydroelectric power.

### Fermentation Room System

In one embodiment, the fermentation reactor utilized in the subject methods can comprise a large, moisture-sealed, enclosed space, having four vertical walls, a floor and a ceiling. The walls can optionally comprise one or more windows and/or doors. This “fermentation room” can replicate the environment that would exist in, for example, a proofing oven fermentation reactor, yet on a much larger scale.

In one embodiment, the fermentation room is fixed onto a portable platform, such as a trailer with wheels.

In one embodiment, the interior walls of the fermentation room have a plurality of horizontal surfaces, upon which the containers for holding inoculated substrate can be placed.

In one embodiment, the surfaces are in the form of shelves. The shelves can be fixed onto the walls of the enclosure. Shelving units can be suspended from the ceiling and/or fixed to the floor.

In one embodiment, the fermentation room comprises a plurality of metal sheet pan racks. The sheet pan racks preferably comprise a plurality of slides for holding trays into which the solid substrate and microbe culture are spread. In one embodiment, the racks are portable, meaning fixed with wheels.

In one embodiment, the pan rack can hold from 10 to 50 trays. Preferably, the slides are spaced at least 3 inches apart from one another to allow for optimal air circulation between each tray.

In one embodiment, the ceiling of the room can optionally be accommodated to allow for air flow, for example, with ceiling vents and/or air filters. Furthermore, the ceiling and walls can be fitted with UV lights to aid in sterilization of air and other surfaces within the system. Advantageously, the use of metal trays and metal pan racks enhances reflection of the UV light for increased UV sterilization.

The room can be equipped with temperature controls, though preferably, the circulation of air throughout the room provides the desired fermentation temperature.

The dimensions of the fermentation room can be customized based on various factors, such as, for example, the location of the room and the number of trays to be placed therein. In one embodiment, the height of the ceiling is at least 8 feet, and the area of the floor is at least 80 square feet.

### Microbial Bioaugmentation Cocktail

In certain embodiments, the subject invention provides microbe-based products comprising one or more microorganisms and/or one or more microbial growth by-products for use in treatment of food processing waste matter, wherein the cell concentration in the product is about 1×10⁶ to 1×10¹³ cells (or CFU) per gram, or higher. In one embodiment, the composition comprises a matrix substrate containing the microorganism and/or the metabolites produced by the microorganism and/or any residual nutrients.

The product of fermentation may be used directly without extraction or purification. If desired, extraction and purification can be achieved using standard extraction methods or techniques known to those skilled in the art.

Upon harvesting of the matrix substrate, microbe, and/or by-products, the product can be dissolved in water to form a liquid product.

Alternatively, upon harvesting of the matrix, microbe and/or by-products, the product can be blended, milled and/or micronized and then dried to form a dry product. This dried product can be dissolved in water and diluted as necessary.

The microorganisms in the microbe-based product may be in an active or inactive form. In some embodiments, the microbes are in vegetative, spore, mycelial, hyphae, conidia form and/or mixtures thereof. The microbe-based products may be used without further stabilization, preservation, and storage.

The dried product and/or liquid product can be transferred to the site of application via, for example, tanker for immediate use.

In other embodiments, the composition can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In certain embodiments the containers are 2 gallons, 5 gallons, 25 gallons, or larger.

Upon harvesting the microbe-based composition from the reactors, further components can be added as the harvested product is processed and/or placed into containers (or otherwise transported for use). The additives can be, for example, buffers, carriers, other microbe-based compositions produced at the same or different facility, viscosity modifiers, preservatives, nutrients for microbe growth, tracking agents, pesticides, and other ingredients specific for an intended use.

Advantageously, in accordance with the subject invention, the microbe-based product may comprise the substrate in which the microbes were grown. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

Organisms that can be cultured according to the subject invention can include, for example, yeasts, fungi, bacteria, archaea, protozoa, metazoa and algae that have been sampled and identified from an activated sludge tank.

The microbial cocktail can comprise, for example, different ratios of Bacillus spp. microbes, such as, for example, Bacillus spp. bacteria, including, but not limited to, B. subtilis, B. licheniformis, B. firmus, B. laterosporus, B. megaterium, and B. amyloliquefaciens. In a specific exemplary embodiment, B. amyloliquefaciens NRRL B-67928 is used.

In some embodiments, the microbes can be Pseudomonas spp. bacteria, such as, for example, P. aeruginosa, P. chlororaphis, P. mallei, P. pseudomallei, P. fluorescens, P. alcaligenes, P. mendocina, and P. stutzeri. Advantageously, in the presence of organic waste matter, these microbes produce enzymes, such as proteases, lipases, reductases and amylases, as well as other growth by-products, that are beneficial to the breakdown of the organic matter.

Advantageously, in the presence of waste matter, these microbes produce enzymes, such as proteases, lipases, reductases, and amylases, as well as other growth by-products, that are beneficial to the breakdown of food processing waste matter.

In one embodiment, the bacteria are denitrifying, or nitrate-reducing, bacteria, such as, e.g., Thiobacillus denitrificans, Micrococcus spp. (e.g., M denitrificans, M roseus), Serratia spp., Pseudomonas spp., and/or Achromobacter spp.

In some embodiments, the microorganisms are protozoa and/or metazoa, such as, for example, amoebae, flagellates, ciliates, rotifers, nematodes, and tardigrades. In some embodiments, the microorganisms are yeasts, fungi, or algae.

In one embodiment, the microbial cocktail comprises microbial growth by-products. These can be produced by the microorganisms of the culture, and/or they can be added to the culture prior to its introduction into the wastewater. Growth by-products can include, for example, biosurfactants, enzymes, biopolymers, solvents, acids, proteins, amino acids, carbohydrates and/or other metabolites that can be useful for treatment of wastewater. In one embodiment, the growth by-product is a biosurfactant.

In certain other embodiments, the compositions comprise one or more microbial growth by-products, wherein the growth by-product has been extracted from a microbial culture and, optionally, purified. For example, in one embodiment, the matrix substrate of the subject methods can be blended to form a thick slurry, which can be filtered or centrifuged to separate a liquid portion from a solid portion. The liquid portion, comprising microbial growth by-products, can then be used as-is or purified using known methods.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application (e.g., at a food processing plant). The facility produces high-density microbe-based compositions in batch, quasi-continuous, or continuous cultivation.

The microbe growth facilities of the subject invention can be located at the location where the microbe-based product will be used. For example, the microbe growth facility may be less than 300, 250, 200, 150, 100, 75, 50, 25, 15, 10, 5, 3, or 1 mile from the location of use.

The microbe growth facilities of the subject invention produce fresh microbe-based compositions comprising the microbes themselves, microbial metabolites, and/or other components of the medium in which the microbes are grown. If desired, the compositions can have a high density of vegetative cells or propagules, or a mixture of vegetative cells and propagules.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. The system is efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

In one embodiment, the microbe growth facility is located on, or near, a site where the microbe-based products will be used, for example, within 300 miles, 200 miles, or even within 100 miles. Advantageously, this allows for the compositions to be tailored for use at a specified location. The formula and potency of microbe-based compositions can be customized for a specific application and in accordance with the local conditions at the time of application.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Fermentation of Bacillus Spores

For Bacillus spp. spore production, a wheat bran-based media is used. The media is sterilized in stainless steel steam pans, then sealed with a lid and pan bands. Following sterilization, the pans are inoculated with seed culture and incubated in a proofing oven for 48-72 hours. At the end of fermentation, 1×10¹⁰ spores/g of Bacillus are harvested.

### Example 2— Solid State Fermentation of Bacillus subtilis and Bacillus licheniformis

Bacillus subtilis and Bacillus licheniformis can be cultivated using solid state fermentation methods. The medium comprises only corn flour (partially hydrolyzed corn meal) or wheat bran. Optionally, added nutrients can be included to enhance microbial growth, such as, for example, salts, molasses, starches, glucose, sucrose, etc.

Foil-covered trays are autoclaved prior to inoculation. The culture medium is spread on the trays in a layer about 1 to 2 inches thick. Grooves and/or holes are made in the substrate to increase the surface area of the medium. To increase the speed of growth, i.e., increase the motility of the bacteria and distribution throughout the culture medium, potato extract or banana peel extract can be added to the culture.

Spores of the Bacillus strain of choice are then sprayed onto the surface of the substrate and the trays are placed into a proofing oven. Fermentation inside the proofing oven occurs at a temperature between 32-40° C. Ambient air is pumped through the oven to stabilize the temperature.

The concentration of microbes grown according to this method when dissolved in water can reach at least 5×10⁹ to 5×10¹⁰ spores/ml. The product is then diluted with water in a mixing tank to a concentration of 1×10⁶ to 1×10⁷ spores/ml. Nutrients that can also be added include, e.g., potassium salts (0.1% or lower), molasses and/or glucose (1-5 g/L), and nitrates.

### REFERENCES

Taguchi, K., Yasuda, K., Hanai, Y., Abe, T, Mae, H. “Wastewater Treatment Process.” U.S. Pat. No. 9,994,469 B2, Jun. 12, 2018. (“Taguchi et al.”).

1. A method for improving the treatment of food processing waste present in wastewater, the method comprising:
taking a sample from the wastewater, wherein the sample comprises one or more types of food processing waste matter;
analyzing the sample to determine the identity of the one or more types of waste matter; and
introducing a customized microbial cocktail comprising one or more beneficial microbial species into the wastewater to increase the throughput of treating the waste matter.

2. The method of claim 1, wherein the wastewater is present in an anaerobic digestor, a lagoon, or a body of water into which food processing waste matter has been introduced.

3. The method of claim 1, wherein the food processing waste matter comprises palm oil milling effluent (POME).

4. The method of claim 1, wherein the food processing waste matter comprises acid-whey.

5. The method of claim 1, wherein the food processing waste matter comprises fats, oils and greases (FOG).

6. The method of claim 1, wherein the microbial cocktail comprises one or more facultative anaerobic bacteria capable of producing enzymes and/or other growth by-products for digesting, purifying, decontaminating, and/or removing the food processing waste matter from the wastewater.

7. The method of claim 6, wherein the microbial cocktail comprises bacteria selected from Bacillus subtilis, B. licheniformis, B. firmus, B. laterosporus, B. megaterium, and B. amyloliquefaciens.

8. The method of claim 6, wherein the microbial cocktail comprises bacteria selected from Pseudomonas aeruginosa, P. chlororaphis, P. mallei, P. pseudomallei, P. fluorescens, P. alcaligenes, P. mendocina, and P. stutzeri.

9. The method of claim 6, wherein the microbial cocktail comprises one or more nitrate-reducing bacteria (NRB), and wherein the method results in denitrification of wastewater, and/or removal of nitrates and/or ammonium from the wastewater.

10. The method of claim 1, wherein the sample further comprises a microbial community comprising one or more microbial species.

11. The method of claim 10, which further comprises analyzing the microbial community to identify the microbial species present within the microbial community and the population percentage of the species;
determining which microbial species are beneficial, commensal and/or detrimental to treatment of the food processing waste, and further determining whether or not the commensal and/or detrimental species comprise at least 25% of the microbial community, wherein a percentage of commensal and/or detrimental species of 25% or greater means the microbial community is in dysbiosis; and
if the microbial community is in dysbiosis, introducing the microbial cocktail into the wastewater to improve the balance of the microbial community and increase the throughput of wastewater treatment.

12. (canceled)

13. The method of claim 1, further comprising applying one or more microbial growth by-products to the wastewater to further enhance the treatment of the wastewater and/or the activity of the microbial cocktail.

14. The method of claim 13, wherein the microbial growth by-products are biosurfactants selected from glycolipids, lipopeptides, cellobiose lipids, flavolipids, phospholipids, lipoproteins, lipopolysaccharide-protein complexes, and polysaccharide-protein-fatty acid complexes.

15. (canceled)

16. The method of claim 14, wherein the biosurfactants are sophorolipids.

17. The method of claim 1, wherein nitrous oxide and/or methane emissions from the aerobic digester, lagoon and/or body of water are reduced.

18. (canceled)

19. The method of claim 1, wherein the number of sulfate-reducing bacteria (SRB) in wastewater is reduced.

20-31. (canceled)
